(19) United States
(12) Patent Application Publication
(10) Pub. No.: US 2026/0054088 A1
(43) Pub. Date: Feb. 26, 2026

(54) SYSTEMS AND METHODS RELATED TO VULVAR PHOTOTHERAPY

(71) Applicant: SARAH MILLER, ST. PETERSBURG, FL (US)

(72) Inventor: SARAH MILLER, ST. PETERSBURG, FL (US)

(21) Appl. No.: 19/188,441

(22) Filed: Apr. 24, 2025

Related U.S. Application Data
(60) Provisional application No. 63/686,904, filed on Aug. 26, 2024.

(51) Int. Cl. A61N 5/06

(52) U.S. Cl. CPC A61N 5/0613; A61N 2005/0645; A61N 2005/0652; A61N 2005/0663

(57) ABSTRACT

Systems and methods according to the present invention relate generally to vulvar phototherapy methods and devices. A device according to the present invention includes a semi-adhesive pad and wedge, which may be aligned with an afflicted area of a patient to administer LED red and/or blue-light phototherapy. The LED lights are generally powered by a central battery, which is preferably rechargeable.

### BACKGROUND OF THE INVENTION

Phototherapy, also known as light therapy, has been a known treatment option for people suffering from a variety of ailments. Phototherapy involves exposing some portion of a patient's body to light waves for a set amount of time, usually as prescribed by a therapist. The light may be provided by a man-made device, or even naturally from the sun. Sometimes, phototherapy is enhanced using medication to target specific conditions such as psoriasis, eczema, mycosis fungoides, itches, vitiligo, newborn jaundice, and morphea.

Several different types of phototherapy have been invented to achieve specific goals. For example, broad band UVB is a type of phototherapy that uses a large (or broad) spectrum of type B ultraviolet light, which has a medium wavelength that reaches only the top layer of the skin. Narrow band UVB uses a smaller portion of the type B UV light spectrum, generally for the same purposes as broad band UVB. Phototherapy may also be conducted using UVA, which uses type A UV light with a longer wavelength to treat conditions that affect deeper regions of the skin. PUVA photochemotherapy uses medication called psoralen to make the skin more sensitive to the UVA light. Such medication may be taken as a pill or applied topically to patient skin. Other forms of phototherapy may use an excimer laser to target a specific bodily area, such as the scalp.

Recent breakthroughs have made phototherapy more cost effective for the average person by using UV light emitted by Light Emitting Diodes (LEDs). These types of phototherapy include blue light therapy (e.g., application of light having wavelengths of about 425 nanometers to about 500 nanometers), and red light therapy (e.g., application of light having wavelengths of about 600 nanometers to about 1000 nanometers, including infrared treatments). Blue light therapy is most commonly used to treat conditions like jaundice, while red light therapy has been more commonly applied to areas of the body to in an attempt to reduce wrinkles, redness, acne, and scars.

Some of the possible drawbacks to phototherapy include skin redness or heat, inflammation, burns or blisters similar to a sunburn, dry skin, nausea, increased wrinkles or fine lines, and the possible risk of developing skin cancers such as basal cell carcinoma, squamous cell carcinoma, and melanoma. Prolonged use of intense phototherapy raises these risks.

Most recently, wearable devices have become available for in office or at home phototherapy sessions. Such devices are generally shaped to target a specific area of the human body. Some of these devices included spatially arranged and distinct LEDs to emit electromagnetic radiations in the red and/or infrared frequencies. These devices may even include rechargeable batteries. However, many of these devices are mostly impractical for repeated use, as they may have inefficient designs and placement of the LEDs, be poorly made or indurable for long-term use, or require bulky and/or impractical fastening arrangements, such as straps, buttons, or loop and hooks. These problems are only exacerbated when the phototherapy device is designed for a sensitive area of the human body.

Therefore, a more efficient, comfortable, cost-effective, durable phototherapy device is sought in the art.

### SUMMARY OF THE INVENTION

Embodiments of systems and methods according to the present invention relate generally to methods and devices for vulvar phototherapy.

An embodiment of a device according to the present invention includes a flexible pad the pad supporting a plurality of electrically powered LED lights to cast light on a patient skin surface that is in contact with or proximate to the pad. Preferably, the device includes a wedge disposed on the pad, the wedge extending along a longitudinal direction of the pad and having a maximum height at a posterior end thereof and a minimum height at an opposite anterior end thereof. The wedge is configured to rest in a vaginal vestibule of a patient, generally extending a majority of a distance between the patient's labial frenulum and clitoral frenulum. Most preferably, the wedge is positioned substantially between the patient's labia minora, such that the entire vulvar region is in at least close proximity with, and most preferably in contact with, the pad. Methods according to the present invention relate to periodically (e.g., pulsing on/off for a predetermined or randomized period such as about 0.1 seconds to about a minute) or continuously applying light to a patient's vulva for a predetermined time (e.g., 10 to 30 minutes) and at a predetermined wavelength (e.g., blue light or red light) to reduce effects of conditions such as common benign chronic vulvar conditions such as genitourinary syndrome of menopause, lichen sclerosus, lichen planus, lichen simplex chronicus, and vulvodynia. Such conditions commonly cause inflammation and mild to intense itching, which is reduced by such light application.

According to an aspect of an embodiment of a device according to the present invention, a vulvar phototherapy device includes a pad configured to be worn by a patient adjacent a vulva and a wedge extending from, and formed integrally with, the pad. At least one of the pad and the wedge includes at least one light source, such as a light emitting diode.

According to another aspect of an embodiment of a device according to the present invention, the light source (e.g., LED(s)) is embedded within at least one (but preferably both) of the pad and wedge, but preferably at a depth or in a configuration so as to allow light from the light source to be transmitted through or from the pad and/or wedge to a tissue surface in contact with or proximate to the pad and/or wedge.

According to yet another aspect of an embodiment of a device according to the present invention, at least one of the pad and wedge is formed from a hypoallergenic material (e.g., hypoallergenic silicone rubber).

According to an aspect of an embodiment of a device according to the present invention, a phototherapy device includes a pad configured to be worn by a patient adjacent a vulva, comprising an inner face and an outer face. The pad includes a pair of diverging legs, each extending to a free end (generally in an outwardly extending V-shape) and a substantially flat flap extending opposite the pair of legs. A wedge extends from the inner (vulva-facing) surface, and at least one of the pad and wedge has at least one light source (e.g., a light emitting diode).

According to another aspect of an embodiment of a device according to the present invention, the light source (e.g., LED(s)) is embedded within at least one (but preferably both) of the pad and wedge, but preferably at a depth or in a configuration so as to allow light from the light source to be transmitted through or from the pad and/or wedge to a tissue surface in contact with or proximate to the pad and/or wedge.

According to still another aspect of an embodiment of a device according to the present invention, the flap has diverging sides extending away from the wedge to a closed free end.

According to yet another aspect of an embodiment of a device according to the present invention, the pad further comprising a first wing and a second wing, the wings extending substantially perpendicular to a longitudinal direction extending from the legs to the flap.

According to a further aspect of an embodiment of a device according to the present invention, the surface area of the pad outer face is greater than the surface area of the pad inner face.

According to an aspect of an embodiment of a method according to the present invention, the method includes the step of placing a device between labia minora of a human. After or during the placing step, using the device, light (e.g., at least one of red light and blue light) is projected onto a medial surface of the labia minora for a predetermined amount of time (e.g. 0.1 seconds to fifteen minutes).

According to another aspect of an embodiment of a method according to the present invention, the device includes a wedge including a first plurality of light sources (LEDs) on or in the wedge.

According to still another aspect of an embodiment of a method according to the present invention, the device further includes a pad coupled to (and more preferably integrally formed with) the wedge. The pad extends from a first free end comprising a flap converging towards the wedge to a second free end comprising two legs diverging away from the wedge. A second plurality of light sources may be embedded in the pad, and the method may further include the step of projecting light from the pad onto at least one of a mons pubis and a perineum of the human.

According to yet another aspect of an embodiment of a method according to the present invention, wherein the human has a vulvar region, said vulvar region having at least one condition selected from the group consisting of jaundice, scarring, rhytids (wrinkles), genitourinary syndrome of menopause, lichen sclerosus, lichen planus, lichen simplex chronicus, vulvodynia, eczema, alopecia areata (hair loss), acne, psoriasis, actinic keratosis, rosacea, sun damage, inflammation, mycosis fungoides, vitiligo, pruritus (itching), and/or collagen deficiency.

### BRIEF DESCRIPTION OF THE DRAWINGS

FIG. 1 is a left elevation view of a device according to the present invention.

FIG. 2 is a top plan view of a device according to the present invention.

FIG. 3 is a cross-sectional view of the device of FIG. 2, taken along line 3-3 of FIG. 2.

FIG. 4 is a bottom plan view of a device according to the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

Turning now to the Figures, embodiments of systems and methods related to vulvar phototherapy may be seen. The device 100 is designed to be worn by patients to provide phototherapy to sensitive areas, most specifically to the patient's vulva to reduce effects of common benign chronic vulvar conditions such as genitourinary syndrome of menopause, lichen sclerosus, lichen planus, lichen simplex chronicus, and vulvodynia. Such conditions commonly cause inflammation and mild to intense itching, which may be treated through the use of phototherapy.

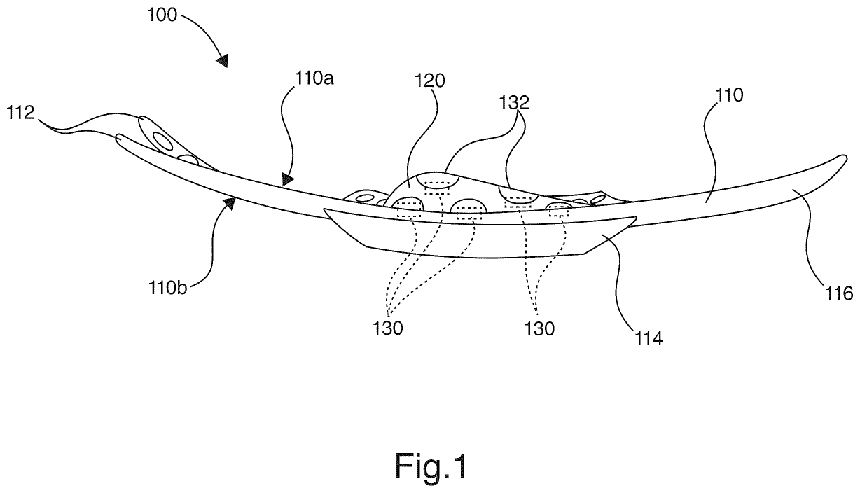

Since the device 100 may be worn by a patient for an extended period during a phototherapy session, but at least because such device 100 is adapted for use adjacent a vulva, it is important that the device 100 is comfortable for the patient to wear. However, the device 100 must also be able to effectively provide phototherapy sessions for the patient. Therefore, a design to balance patient comfort and therapy effectiveness is desired and achieved by the device 100. As seen in FIG. 1, an embodiment of a system according to the present invention may include a device 100 comprising a wedge 120, preferably formed integrally with a pad 110, wherein at least one (but preferably both) of the wedge 120 and pad 110 include at least one light source, such as LED lights 130, which may be embedded therewithin.

For optimal patient comfort, the pad 110 is preferably formed from a soft, smooth, pliable, durable, and hypoallergenic material such as silicone rubber to provide maximum comfort with minimal chance of skin irritation for the patient and further to provide sufficient light transmission characteristics. Silicone is relatively easy to mold into a desired form (or even a customized form) and holds its shape once it is set. Further, objects may be placed or embedded in silicone in the molding process, such as the LED lights 130 embedded in the pad 110 and wedge 120. A preferred silicone will combine adequate light transmission and comfort considerations, preferably including greater than 90% visible light transmission (at 2 millimeter thickness of material extending over the LEDs 130) and a Shore A hardness of preferably between 5 and 60, with a more preferred Shore A hardness of between about 5 and about 40, with a most preferred Shore A hardness of between about 10 to about 40.

Much like the material, the shape of the device 100 plays a role in patient comfort. The pad 110 preferably comprises an outer face 110b and inner face 110a, along with several areas designed to create a comfortable and snug fit with a portion of a patient body (e.g., a vulva), with each area acting to help temporarily adhere the device 100 to the patient during a phototherapy session. For example, the pad 110 preferably includes a pair of legs 112 extending in an at least substantially co-planar (e.g., substantially co-curviplanar) fashion, which diverge from each other to form a substantially U or V shape, each to a free end. In use, these legs 112 are preferably formed to extend approximately along a gluteal fold or inguinal crease of the patient to help hold the device 100 in place. The legs 112 may include a non-reactive adhesive coating or similar means to temporarily adhere to the body of the patient. More preferably, however, the pad 110 is created from material that is itself slightly adhesive, which may temporarily stick to the user's body without the need of outside adhesive. Additionally or alternatively, the pad 110 may be held in place with (or even supported by or attached to) a pair of underwear.

The pad 110 further preferably extends opposite the legs 112 into a flap 116, preferably formed integrally with the rest of the pad 110, and preferably protruding co-planarly (e.g., substantially co-curviplanarly) with diverging sides and a closed free end. Most preferably, the flap 116 projects in a direction opposite the legs 112. Like the rest of the pad 110, the flap 116 is most preferably formed of a slightly adhesive material, such that it may temporarily adhere to or be supported against a patient's mons pubis during use.

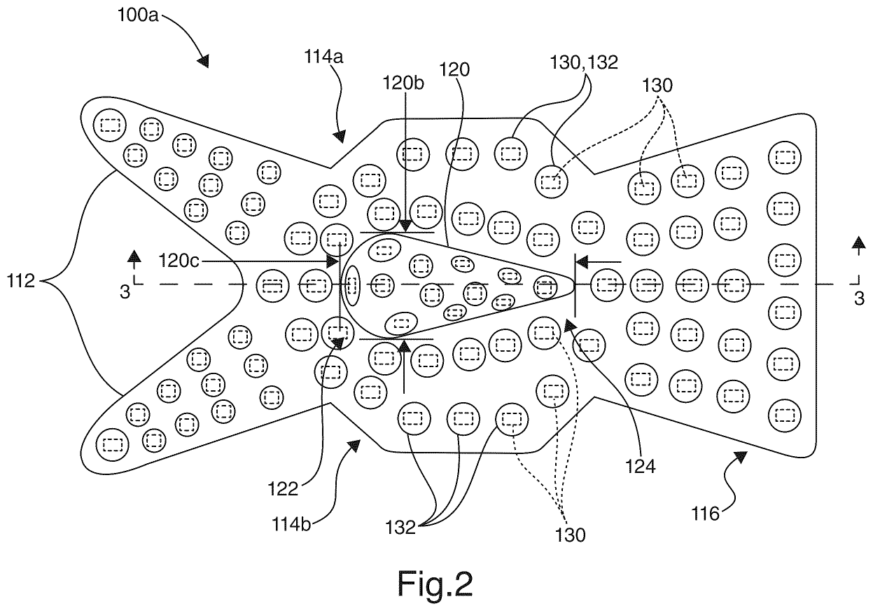

The pad 110 may also optionally comprise a pair of wings 114a-b, one wing 114a on an opposite side of the pad from the other wing 114b, as seen in FIG. 2. The wings 114a-b may take varying shapes based on the embodiment, generally extending between a leg 112 and the flap 116. In the embodiment depicted in FIG. 2, the wings 114a-b are preferably trapezoidal in shape, extending from the pad 110 and tapering in width. Like the legs 112 and flap 116, the wings 114a-b are preferably slightly adhesive and serve to temporarily bond the pad 110 to the patient's skin while the device 100 is in use. While some embodiments of the pad 110 may be substantially flat (in free space), other embodiments, such as the one shown in FIG. 1, are preferably slightly curved in free space. For example, the inner face 110a of the pad 110 may have slightly less surface area than the outer face 110b, which forces the extremities (legs 112 and flap 116) to slightly curl towards the inner face 110a. This slight curl design helps the legs 112 and flap 116 to conform to the patient's skin during use, providing a more stable and comfortable experience for the patient.

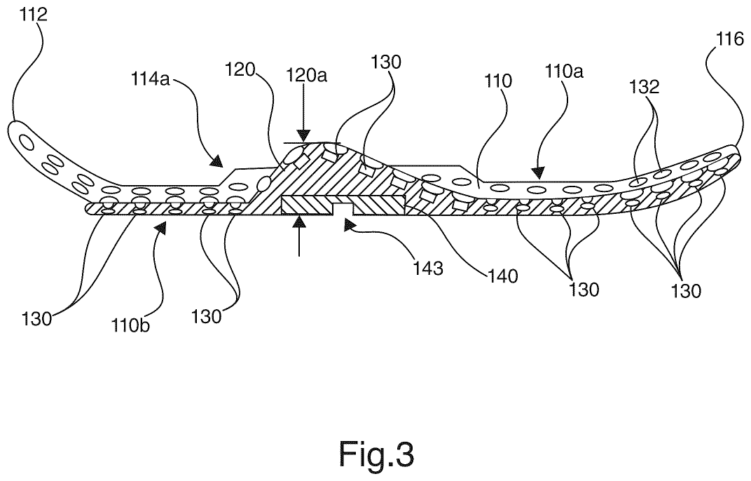

Located between the opposing legs 112 and flap 116, and also between opposing wings 114a-b, the wedge 120 preferably protrudes from the inner face 110a of the pad 110. Generally speaking, the wedge 120 is substantially half-teardrop shaped extending from the inner surface 110a, the wedge 120 having a height 120a and width 120b that taper along a wedge length 120c from a wider and taller (“WT”) end 122 closer in proximity to the legs 112 to a narrow and shorter (“NS”) end 124 closer in proximity to the flap 116, as seen in FIGS. 2-3. The wedge 120 has a curved upper surface, with the radius of such surface at the WT end 122 being greater than the radius at the NS end 124.

Much like the rest of the pad 110, the wedge 120 is preferably made of a soft, smooth, pliable, durable, and hypoallergenic material, such as silicone, which may be and preferably is the same type of silicone used for the pad 110. However, the wedge 120 preferably is adhesive-free during use. Instead, the wedge 120 is preferably able to resist adherence to the patient's skin during proximity and contact, allowing slight movement of the wedge 120 during device 100 use to ensure all areas of the patient currently being treated are adequate covered. The wedge 120 is also preferably slightly less pliable than (e.g., having a greater Shore A hardness rating) the rest of the pad 110, such that the wedge 120 will not be substantially deformed by contact with the patient's skin during use.

In use, the wedge 120 is most preferably aligned within the vaginal vestibule of a patient, generally extending a majority of a distance between the patient's labial frenulum and clitoral frenulum. Most preferably, the wedge 120 is positioned substantially between the patient's labia minora, such that the entire vulvar region is in at least close proximity with, and most preferably in contact with, the pad 110 and/or wedge 120. The light sources are activated before, during or after placement of the pad and/or wedge against the vulva, and may be controlled with a switch to directly selectively interrupt electrical power, or to activate an electronic controller (e.g., microprocessor or ASIC) operative coupled to a drive circuit to provide power to the light sources. The light sources may be operated continuously, periodically, pseudorandomly, and/or in varying intensities, for or throughout a predetermined period of time, such as 0.1 seconds to about 30 minutes, and then deactivating the light sources for at least an equal predetermined period of time.

Phototherapy is accomplished using operatively interconnected (e.g., with wires) LED lights 130 embedded and/or placed within the material of the pad 110 and wedge 120. The LED lights 130 are most preferably red and/or blue lights, with some embodiments of the present invention featuring a mix of these colors and other embodiments featuring one color only. In manufacturing the device 100, the LED lights 130 (and their interconnections) are placed within the material of the pad 110 and wedge 120 before the material (e.g., liquid silicone) sets, or the LEDs 130 (and wires) may be positioned within a mold in predetermined locations and then the encapsulant material (e.g., the liquid silicone) may be poured over and around them. Alternatively, LED lights 130 may be placed after manufacturing of the rest of the device 100. Specific light designs may vary based on embodiments, such as rows or concentric circles, however it is preferable that each area of the pad 110 (including the legs 112, wings 114a-b, and flap 116) include the LED lights 130 as well. The device 100 is most preferably able to substantially cover the entire treatment area of the patient, therefore the LED lights 130 preferably provide light through a substantial portion of the inner face 110a of the pad 110. However, embodiments of the present invention wherein certain portions of the pad 110 do not comprise LED lights 130 (or the LED lights 130 provided thereon may be selectively activated or deactivated) are contemplated as well. Such embodiments may be used to target specific patient areas, while leaving other areas purposefully untreated. For instance, LED lights 130 may be provided or activated only within the wedge 120 and not in the pad 110, or vice versa.

While the LED lights 130 are preferably embedded within the pad/wedge material (e.g., the LEDs are set approximately 2-4 mm below and covered by the inner surface 110a), the LED lights 130 may alternatively shed light onto the patient's treatment area through a series of holes 132 in the inner face of the pad 110, such that the light delivery is not hindered by the pad 110 material. The diameter of each hole 132 is preferably substantially the same, however some embodiments of the current invention may have holes 132 of different diameters depending on the intended purpose and/or type of phototherapy being applied. However, as indicated, preferred materials do not affect effective light transmission therethrough. In such embodiments, the inner face 110a of the pad 110 may be substantially solid and blue and/or red lights from the LED lights 130 simply pass through the inner face 110a before skin exposure.

As stated above, the LED lights 130 are preferably blue and/or red in color. Using LED lights is advantageous for phototherapy treatment, as LED lights produce almost negligible heat and no known harmful radiation. They are also preferable to the use of lasers, which could cause unwanted damage and scarring to the skin. Phototherapy using LED lights is useful for treating a variety of conditions, including eczema, hair loss, acne, psoriasis, actinic keratosis, rosacea, sun damage, inflammation, and wrinkles. Blue-light LED phototherapy is specifically useful for treating superficial conditions, and skin damage, such as acne. Red-light travels further into the skin to reach more subdermal conditions, such as inflammation and collagen production. Whether used together or separately, red and blue-light LED phototherapy have significant phototherapeutic applications for treating patients with skin conditions and damage.

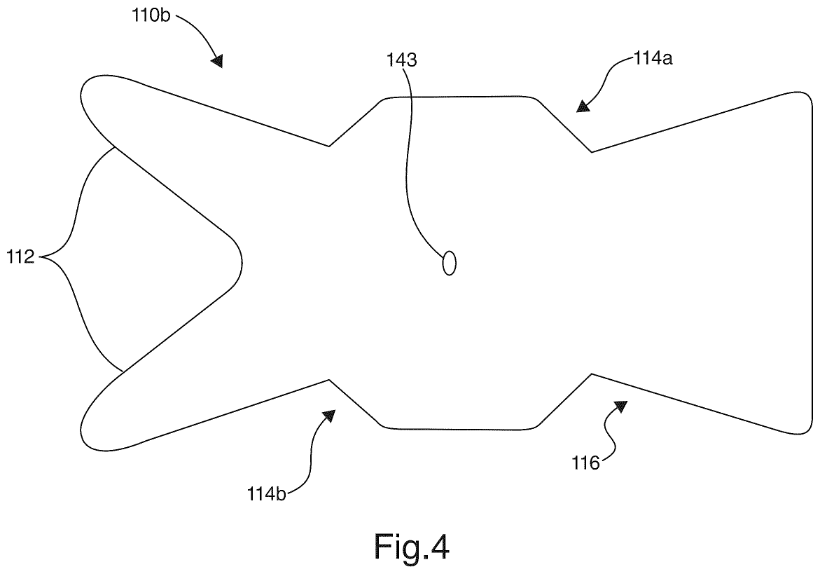

In some embodiments of the present invention, each LED light 130 may be individually battery powered. However, replacing every battery would be impractical. Therefore, a central battery 140 is preferably located within the wedge 120, which may power all of the LED lights 130 (which are preferably electrically interconnected in parallel) at the same time, as seen in FIG. 3. The central battery 140 is preferably programmable, or its output selectable, such that the intensity of the LED lights 130 is adjustable based on the therapy prescribed for the patient by a healthcare professional, the device 100 manufacturer, or based on the patient's own intended use. Most preferably, the central battery 140 is rechargeable and may be connected to an external power source through a cable (e.g. USB-C cable) to charge through a charging port 143 located on the outer face 110b of the pad 110, seen in FIG. 4. Such a rechargeable battery is well known in the art and commercially available. Alternatively, a battery may be remotely mounted, such as in an electronics module or battery pack that may further include a programmed microcontroller that may be programmed to execute a predetermined light on/off timing pattern. The electronics module may be mounted and supported, such as on or in a pair of underwear or panties and coupled to the LEDs with one or more wires, for example.

The foregoing is considered as illustrative only of the principles of the invention. Furthermore, because numerous modifications and changes will readily occur to those skilled in the art, it is not desired to limit the invention to the exact construction and operation shown and described. While the preferred embodiment has been described, the details may be changed without departing from the invention.

I claim:

1. A vulvar phototherapy device comprising:
a pad configured to be worn by a patient adjacent a vulva; and
a wedge extending from, and formed integrally with, the pad,
wherein at least one of the pad and the wedge comprises at least one light source.

2. The device of claim 1, wherein the light source is an LED.

3. The device of claim 2, wherein the LED is embedded within at least one of the pad and wedge.

4. The device of claim 1, wherein both the pad and wedge comprise at least one light source.

5. The device of claim 1, wherein at least one of the pad and wedge comprise hypoallergenic material.

6. The device of claim 5, wherein the hypoallergenic material is silicone rubber.

7. A phototherapy device comprising:
a pad configured to be worn by a patient adjacent a vulva, comprising an inner face and an outer face, the pad comprising:
a pair of diverging legs, each extending to a free end;
a substantially flat flap extending opposite the pair of legs; and
a wedge extending from the inner face of the pad,
wherein at least one of the pad and wedge comprise at least one light source.

8. The device of claim 7, wherein the light source is an LED.

9. The device of claim 8, wherein the LED is embedded within at least one of the pad and wedge.

10. The device of claim 7, wherein both the pad and wedge comprise at least one light source.

11. The device of claim 7, wherein the pad and wedge comprise hypoallergenic material.

12. The device of claim 11, wherein the hypoallergenic material is silicone rubber.

13. The device of claim 7, wherein the flap has diverging sides extending to a closed free end.

14. The device of claim 7, the pad further comprising a first wing and a second wing, the wings extending substantially perpendicular to a longitudinal direction from the legs to the flap.

15. The device of claim 7, wherein the surface area of the pad outer face is greater than the surface area of the pad inner face.

16. A method comprising the steps of:
placing a device between labia minora of a human; and
using the device, projecting light onto a medial surface of the labia minora for a predetermined amount of time.

17. The method of claim 16, wherein the device comprises:
a wedge; and
a first plurality of light emitting diodes embedded in the wedge.

18. The method of claim 17, wherein the device further comprises a pad coupled to the wedge, the pad extending from a first free end comprising a flap converging towards the wedge to a second free end comprising two legs diverging away from the wedge; a second plurality of light emitting diodes embedded in the pad, the method further comprising the step of:
projecting light from the pad onto at least one of a mons pubis and a perineum of the human.

19. The method of claim 16, wherein the predetermined amount of time is selected within the range of 0.1 seconds to fifteen minutes.

20. The method of claim 16, wherein the light is at least one of red light and blue light.
